(19) United States
(12) Patent Application Publication
(10) Pub. No.: US 2024/0131108 A1
(43) Pub. Date: Apr. 25, 2024

(54) AMINO ACID-BASED GLASS, PREPARATION METHOD AND USE THEREOF

(71) Applicant: INSTITUTE OF PROCESS ENGINEERING, CHINESE ACADEMY OF SCIENCE, Beijing (CN)

(72) Inventors: Xuehai YAN, Beijing (CN); Ruirui XING, Beijing (CN); Chengqian YUAN, Beijing (CN)

(21) Appl. No.: 18/264,967

(22) PCT Filed: Sep. 29, 2021
(86) PCT No.: PCT/CN2021/121578

§ 371 Date: Aug. 10, 2023

(51) Int. Cl. A61K 38/05; A61K 6/836; A61K 31/198; C08L 89/00

(52) U.S. Cl. CPC A61K 38/05; A61K 6/836; A61K 31/198; C08L 89/00

(57) ABSTRACT

The present invention discloses biodegradable glass based on an amino acid, a peptide and a derivative, as well as the preparation method and use thereof. The main raw material of the glass is one or more of an amino acid, a peptide, a derivative or salt thereof. Compared with traditional glass, the glass of the present invention has significant advantages such as high biocompatibility, biodegradability, being 3D printable, and being compostable, etc., and its preparation process is simple and green, which can effectively avoid the influence of the traditional glass on the ecological environment. The glass of the present invention has a wide range of applications in the fields such as medicine, building material, chemical industry, food, electronics, national defense, etc., including but not limited to tissue engineering, tooth/bone repair, drug sustained-release, cell/protein sequestration, optical fiber communication, coatings, precision instruments, etc.

### CROSS REFERENCE

This application is a Section 371 National Stage Application of International Application No. PCT/CN2021/121578 filed on Sep. 29, 2021, the content of which is incorporated herein by reference in its entirety.

### FIELD OF THE INVENTION

The present invention relates to a glass material as well as the preparation method and use thereof, in particular to amino acid-based biomolecular glass as well as the preparation method and use thereof, and belongs to the field of new materials.

### BACKGROUND OF THE INVENTION

Glass is generally prepared from inorganic minerals such as silica, calcium carbonate, etc. as the main raw material, and is one of the most commonly used materials in daily life. Glass is hardly degradable under natural conditions and is easily broken. Therefore, in terms of pollution, hazard, or permanence, the impact of glass on the environment and ecology is significant.

Currently, a variety of glass materials, articles and their manufacturing methods have been disclosed. For example, a method for manufacturing silicate glass, silicate glass, and a silica raw material for silicate glass have been disclosed (see WO2015/129495 JA; 20150903); a glass product using β-quartz or β-spodumene solid solution as the main raw material has been disclosed (see WO2005/058766 EN; 20050630); lithium silicate glass ceramic and lithium silicate glass containing divalent metal oxide have been disclosed (see WO2013/053864 DE; 20130418).

It is worth noting that the bioglass invented by L. L. Hench of the University of Florida in 1969 is mainly composed of 45% Na₂O, 25% CaO, 25% SiO₂, and 5% P₂O₅. The exemplary compositions and applications of bioglass (also known as bioactive glass) have been disclosed (see U.S. Pat. Nos. 4,478,904A; 6,338,751B1; 7,569,105B2).

The above disclosed glass materials and articles have in common that the raw materials are all inorganic minerals. Amino acid-based biomolecular glass materials and preparation methods thereof have not been disclosed so far.

An amino acid is the basic unit for constituting a protein, and a peptide is a compound formed by linking two or more amino acids via peptide bonds. Amino acids and peptides are important components of living organisms, and play extremely important roles in information transmission, metabolism, disease, and aging, etc. of living organisms. Amino acid-based biomolecules have a very high biocompatibility, and have a definite metabolic mechanism in the organisms and are biodegradable. Unexpectedly, the present inventors have found that biodegradable glass with a glassy state structure at room temperature can be obtained from amino acids, peptides, and their derivatives through a specific preparation process, and based on this finding, the present invention is completed. The amino acid-based biomolecular glass obtained according to the present invention has a prospect to be widely used as a new material in the fields of medicine, building material, chemical industry, food, electronics, national defense, etc.

### SUMMARY OF THE INVENTION

A primary object of the present invention is to provide amino acid-based biomolecular glass and its preparation method. Such glass is environmentally friendly, has high biocompatibility, is biodegradable, 3D printable, and compostable, and has a simple and green preparation process.

In a first aspect, the above-mentioned amino acid-based glass is characterized in that, its main raw material is an amino acid represented by formula (1), a peptide, and derivatives thereof, and the content of the main raw material in the glass is 70 wt % or more, preferably 80 wt % or more, further preferably 90 wt % or more,

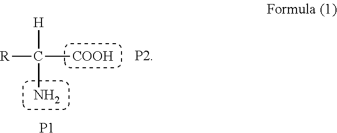

The amino acid includes glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, selenocysteine, and pyrrolysine.

The peptide is a molecule formed by condensation of n amino acids via peptide bonds, where n≥2, preferably 2≤n≤10.

The derivatives are amino acids or peptides having protecting group(s) for the amino group P1 and the carboxyl group P2, wherein:
the protecting group at P1 includes, but not limited to, Trt, Boc, Fmoc, Cbz/Z, Allyl, C₂-C₁₈acyl, benzoyl, and naphthoyl; and the protecting group at P2 includes, but not limited to, OFm, Otbu, OBzl, OAll, OMe, and OEt; and
it is protected individually or simultaneously at P1 and at P2.

The derivatives also include the molecules, isomers, and salts thereof having similar structural skeletons to the above-mentioned amino acid molecules, peptide molecules, or their derivative molecules.

In a second aspect, the above-mentioned amino acid-based glass is characterized in that, it is completely prepared from the above-mentioned amino acids, peptides and derivatives thereof.

In a third aspect, the above-mentioned amino acid-based glass is characterized in that:
the amino acid-based glass can be prepared from a single molecule, including a single amino acid molecule, a single peptide molecule, a single amino acid derivative, or a single peptide derivative; or
it can also be prepared from a combination of two or more of the above-mentioned molecules, including a combination of amino acid molecules, a combination of peptide molecules, a combination of amino acid derivative molecules, a combination of peptide derivative molecules, a combination of amino acid molecules and peptide molecules, a combination of amino acid molecules and amino acid derivative molecules, a combination of amino acid molecules and peptide derivative molecules, a combination of peptide molecules and amino acid derivative molecules, a combination of peptide molecules and peptide derivative molecules, a combination of amino acid derivative molecules and peptide derivative molecules, a combination of amino acid molecules, peptide molecules and amino acid derivative molecules, a combination of amino acid molecules, peptide molecules and peptide derivative molecules, a combination of amino acid molecules, amino acid derivative molecules and peptide derivative molecules, and a combination of amino acid molecules, peptide molecules, amino acid derivative molecules and peptide derivative molecules.

In a fourth aspect, the above-mentioned amino acid-based glass is characterized in that, in addition to the above-mentioned main raw material, the amino acid-based glass may further comprise auxiliary raw materials, including one of or a mixture of two or more selected from clarifiers, fluxes, opacifiers, and colorants, wherein
the proportion of the auxiliary raw materials is 0˜5 wt %, preferably 0˜1 wt %;
the clarifier includes one of or a mixture of two or more of antimony oxide, sodium nitrate, ammonium nitrate, sodium sulfate, calcium sulfate, sodium chloride, and ammonium chloride;
the flux is one of or a mixture of two or more of sodium carbonate, potassium carbonate, sodium carbonate, and potassium nitrate;
the opacifier is one of or a mixture of two or more of cryolite, sodium fluorosilicate, and tin phosphide;
the colorant is a metal compound of transition elements such as cobalt, manganese, nickel, iron, copper, etc.

In a fifth aspect, a method for preparing amino acid-based glass comprises the steps of:
raising the temperature of raw materials to a temperature higher than the melting point under an inert gas atmosphere, and maintaining at this temperature for a period of time,
lowering the temperature to below room temperature, and
transferring the temperature-lowered sample to an annealing furnace for annealing treatment.

In a preferred embodiment of the present invention, the temperature higher than the melting point (Tₘ) is a temperature higher than the melting point by 5˜200 K, preferably higher than the melting point by 10˜50 K; and the time for maintaining is 5 min˜1 h, preferably 15˜30 min.

In a preferred embodiment of the present invention, the temperature for the annealing treatment is a temperature lower than the glass transition temperature (T_{g}) by 20˜100 K, preferably 20˜50 K; and the time for the annealing treatment is 5 min˜3 h, preferably 15 min˜1 h.

In one embodiment of the present invention, the amino acid-based glass is single-molecule glass, prepared by the steps of:
(1) weighing an amount of powders of an amino acid, a peptide, or derivatives thereof, grinding in a mortar uniformly, and transferring to a crucible;
(2) placing the crucible containing the raw material from step (1) in a heating device under an inert gas atmosphere;
(3) heating the crucible from room temperature to a temperature M₁ at a heating rate S₁ by conducting a heating treatment on the device from step (2), and maintaining at this temperature for a period of time T₁, wherein:
   S₁ is 1˜50 K min⁻¹, preferably 2˜10 K min⁻¹;
   M₁ is a temperature higher than T. by 5˜200 K, preferably higher than T. by 10˜50 K; and
   T₁ is 5 min˜1 h, preferably 15˜30 min;
(4) lowering the temperature of the crucible to a temperature M₂ at a cooling rate S₂ by conducting a cooling treatment on the device from step (3), wherein:
   S₂ is 1˜100 K min⁻¹, preferably 50˜100 K min⁻¹; and
   M₂ is 273.15 K (the temperature of an ice-water mixture) or 293.15˜298.15 K (room temperature/normal temperature); and
(5) transferring the sample from step (4) to an annealing furnace at a temperature M₃, and maintaining at this temperature for a period of time T₃ for conducting an annealing treatment on the glass, wherein:
   M₃ is a temperature lower than T_{g} by 20˜100 K, preferably lower than T_{g} by 20˜50 K; and
   T₃ is 5 min˜3 h, preferably 15 min˜1 h.

If it is a glass formed by a combination of two or more molecules, the process includes the following modified steps:
(1° weighing the powders of each component separately, grinding in an respective mortar uniformly, and then transferring all components to different crucibles respectively;
(2° conducting the above-mentioned steps (2) and (3);
(3° mixing the melted components at a certain ratio in the same crucible, and stirring them uniformly, wherein the ratio for mixing is preferably 1:1: . . . ;
(4° maintaining the mixture obtained from step(3°) at the temperature M₁ for a period of time Tₛ; and)
(5° conducting the above-mentioned steps (4) and (5); or the following steps:
(6° weighing the powders of each component separately, mixing at a certain ratio and stirring them uniformly, wherein the ratio for mixing is preferably 1:1: . . . ;
(7° grinding the uniformly-mixed powders in a mortar uniformly, then transferring to a crucible; and)
(8° conducting the above-mentioned steps (2) through (5).

In a sixth aspect, a method for preparing amino acid-based glass is characterized in that, in addition to the main raw material, auxiliary raw materials are added, and the steps are as follows:
(1) weighing the main raw material and the auxiliary raw materials, mixing at a certain ratio and stirring them uniformly, and transferring to a crucible; and
(2) conducting the above-mentioned steps (2) through (5) of the fifth aspect.

In a seventh aspect, the Tₘ and T_{g} are measured by a standard differential scanning calorimetry (DSC) method, comprising:

setting the heating rate of DSC, preferably at 10 K min⁻¹, and drawing a curve with the temperature as the abscissa and the heat flow as the ordinate, recording the initial temperature and the termination temperature for sample melting, and taking the midpoint temperature of the initial temperature and the termination temperature as Tₘ;
upon raising to a temperature higher than Tₘ by 20 K, maintaining at this temperature for 10 min;
setting the cooling rate of DSC, preferably at 10 K min⁻¹, and upon cooling to 273.15 K, maintaining at this temperature for 10 min;
conducting a second temperature rise, setting the heating rate of DSC, preferably at 10 K min⁻¹, and drawing a curve with the temperature as the abscissa and the heat flow as the ordinate, recording the initial temperature and the termination temperature of the glass transition by extrapolating tangent, and taking the midpoint temperature between the initial temperature and the termination temperature as T_{g}.

Amino acid-based glass which is prepared by the above method is disclosed.

In an eighth aspect, the amino acid-based glass of the present invention and its preparation method have the following advantages and beneficial effects:
(1) The amino acid-based glass of the present invention has properties such as hard texture, brittleness, transparency, light transmittance, etc.: the hardness is between 420˜550 HV, preferably between 500˜550 HV; the transparency is distributed between 30%˜91%, preferably between 80%˜91%;
(2) The amino acid-based glass of the present invention has a relatively good glass forming ability (GFA), and the brittleness index (m) of the amino acid-based glass is distributed between 10˜100, preferably between 10˜50;
(3) The amino acid-based glass of the present invention is environmentally friendly, has a high biocompatibility, and is biodegradable;
(4) The preparation process for the amino acid-based glass of the present invention is simple, highly reproducible, green and environmentally friendly;
(5) The amino acid-based glass of the present invention can be used for additive manufacturing (3D printing);
(6) The amino acid-based glass of the present invention can be used for composting, greatly reducing the damage to the ecological environment caused by traditional glass.

In a ninth aspect, the amino acid-based glass of the present invention has the following applications: it is useful in the fields such as medicine, building material, chemical industry, food, electronics, national defense, etc., including but not limited to tissue engineering, tooth/bone repair, drug sustained-release, cell/protein sequestration, optical fiber communication, coatings, precision instruments, etc.

In a tenth aspect, the amino acid-based glass of the present invention can melt drug molecules during the melting process, preferably the drug molecules are drug molecules having a short half-life, and/or insoluble drug molecules.

The drug molecules include any one of or a mixture of two or more of tumor chemotherapy drug molecules, contrast agent molecules, antipyretic, analgesic and anti-inflammatory molecules, traditional Chinese medicine monomers, immunomodulators and other molecules.

The chemotherapy drug molecules include any one of or a mixture of two or more of pemetrexed, fluorouracil, doxorubicin, paclitaxel, docetaxel, vincristine, cisplatin, tamoxifen, megestrol, goserelin, and their analogs.

The contrast agent molecules include any one of or a mixture of two or more of barium sulfate, iodine preparations (sodium iodide, diatrizoate meglumine, iothalamate meglumine, ioxaglic acid, iohexol, iopromide, iopamiro, iotrolan, iodized oil, myodil), ¹⁸FDG, Gd-DTPA, Mn-DPDP, SPIO, and their analogs.

The antipyretic, analgesic and anti-inflammatory molecules include any one of or a mixture of two or more of aspirin, ibuprofen, acetaminophen, indomethacin, nimesulide, rofecoxib, celecoxib, and their analogs.

The traditional Chinese medicine monomer molecules include any one of or a mixture of two or more of curcumin, nobiletin, tripterygium methyl ester, astragalus, versicolor polysaccharide, and their analogues.

The immunomodulators include any one of or a mixture of two or more of glycoprotein, pidotimod, thymosin α1, muramyl dipeptide, interferon γ, interleukin-2, levamisole, and their analogues.

The other molecules are any one of or a mixture of two or more of drugs requiring sustained-release such as insulin, paliperidone, nifedipine, ranitidine hydrochloride, and their analogs.

It is characterized in that, it can be used as a subcutaneous embedding agent, an oral agent, a tissue engineering scaffold material, preferably the raw material is an amino acid, a peptide or their derivatives having a biological activity, and a local and sustained release of drugs can be realized with the biodegradation of the amino acid-based glass.

In an eleventh aspect, the amino acid-based glass of the present invention can melt other functional formulations during the melting process or can be coated on the surface of a glass material in a form of coating to perform a certain function, including but not limited to as an conductive agent, a bactericidal/antiseptic agent, and an anti-radiation agent.

The conductive agent includes any one of or a mixture of two or more of indium tin oxide, graphite, polyacetylene, and their analogues.

The bactericidal/antiseptic agent includes any one of or a mixture of two or more of nano-silver, chlorine preparations, peroxides, organic sulfur, organic bromine, nitrogen and/or sulfur-containing heterocyclic compounds, and their analogues.

The anti-radiation agent includes any one of or a mixture of two or more of melanin, polyimide, and their analogues.

In a twelfth aspect, the amino acid-based glass of the present invention can melt a drug or a functional formulation during the melting process, in which a powder co-melting method can be employed; such a preparation method can also be employed, comprising pre-dissolving the drug or functional formulation in a good solvent, blending with the amino acid-based glass in a melt state, and then removing the solvent, characterized in that:
the content of drug molecules is 0.01˜25 wt %, preferably 0.1˜1 wt %; and
the content of functional molecules is 0.01˜5 wt %, preferably 0.1˜1 wt %.

### BRIEF DESCRIPTION OF DRAWINGS

FIG. 1 shows the physical picture of the Ac-Lys glass prepared in Example 1 at room temperature, which can be processed into glass beads or glass coatings.

FIG. 2 shows the DSC-TGA diagrams of the Ac-Lys glass prepared in Example 1, of which the melting temperature Tₘ=536.70 K, and the weight loss was not significant at the melting point temperature, indicating that Ac-Lys did not decompose when melted at a high temperature.

FIG. 3 shows the DSC diagram of the Ac-Lys glass prepared in Example 1, of which the glass transition temperature T_{g}=295.10 K.

FIG. 4 shows the H-NMR spectrum of the Z-Phe-Phe glass prepared in Example 2. Compared to the Z-Phe-Phe raw material, the peaks did not change significantly, indicating that the chemical composition of the raw material peptide molecules did not change when subjected to heating, melting and annealing treatments.

FIG. 5 shows the light transmittance of the Z-Phe-Phe glass prepared in Example 2, which is comparable to commercially available glass.

FIG. 6 shows the DSC diagram of the Z-Phe-Phe glass prepared in Example 2, of which the glass transition temperature T_{g}=320.75 K.

FIG. 7 shows the picture of Boc-Gly powders and Boc-Gly glass prepared in Example 3 under a polarizing microscope, demonstrating that the glass formed was amorphous.

FIG. 8 shows the test results for the biocompatibility of the Boc-Gly glass prepared in Example 3, in which the glass was processed into a square coating with a width of 2 cm, co-incubated with 3T3 cells, and the cell activity was tested by the MTT method.

FIG. 9 shows the test results for mechanical properties of the Boc-Ala glass prepared in Example 4.

FIG. 10 shows the biodegradation curve of the Boc-Ala glass prepared in Example 4 in a compost soil sample, in which the initial mass of the glass sample was 42.58 mg.

FIG. 11 shows the performance test results for the mixed glass prepared in Example 5.

FIG. 12 shows the degradation of the mixed glass prepared in Example 5 in an artificial gastric juice (following the preparation method of the Chinese Pharmacopoeia).

FIG. 13 shows the body weight change of mice after gastric perfusion of the mixed glass prepared in Example 5. The cycle of gastric perfusion to mice is once every 5 days with a dose of 5 mg kg⁻¹ for an observation period of 30 days, resulting in the number of gastric perfusions of 5.

FIG. 14 shows the pattern printed with a 3D printing equipment from the mixed glass prepared in Example 6, in which the mixed powders were placed in the barrel of the 3D printing equipment, and the heating temperature was set to 450 K.

FIG. 15 shows the degradation of the mixed glass prepared in Example 6 after being implanted in an animal mouse model.

FIG. 16 shows the biodegradation of the insulin-loaded amino acid-based glass prepared in Example 7 after being subcutaneously implanted in mice over time.

FIG. 17 shows the blood glucose change of diabetic mice after oral gastric perfusion of the insulin-loaded amino acid-based glass prepared in Example 7.

### DETAILED DESCRIPTION

The technical solutions of the present invention will be described in detail below by way of examples, but the protection scope of the present invention is not limited thereto.

### Example 1

A method for preparing a lysine-based glass comprises the steps of:
(1) Weighing 20 mg of N-acetyl-L-lysine (Ac-Lys) powders, grinding in a mortar uniformly, then transferring to a crucible;
(2) Placing the crucible containing Ac-Lys powders from step (1) in a heating device under N₂ atmosphere;
(3) Heating the crucible from room temperature to 600 K at a heating rate of 10 K min⁻¹ by conducting a heating treatment on the device from step (2), and maintaining at this temperature for 10 min;
(4) Lowering the temperature of the crucible to a temperature of 273.15 K at a cooling rate of 10 K min⁻¹ by conducting a cooling treatment on the device from step (3); and
(5) Transferring the sample from step (4) to an annealing furnace at a temperature of 283.15 K, and maintaining at this temperature for 20 min for conducting an annealing treatment on the glass, thereby obtaining Ac-Lys glass.

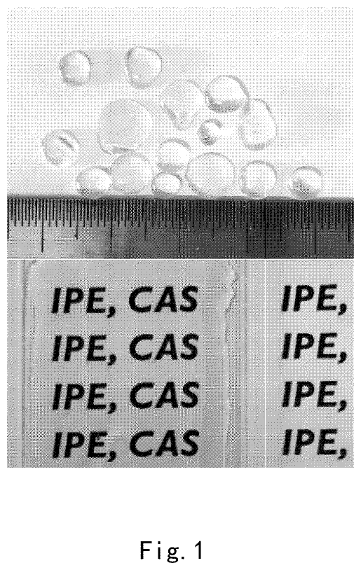

FIG. 1 shows the physical picture of the Ac-Lys glass prepared in Example 1 at room temperature, which can be processed into glass beads or glass coatings.

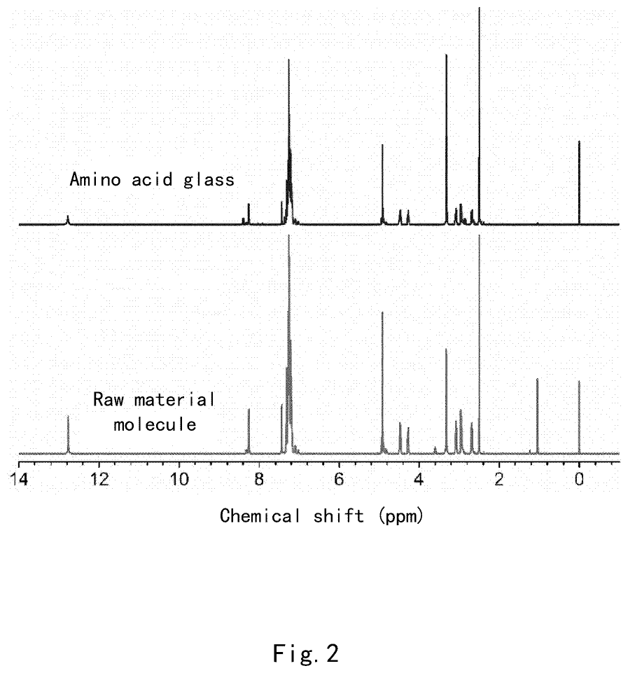

FIG. 2 shows the DSC-TGA diagrams of the Ac-Lys glass prepared in Example 1, of which the melting temperature Tₘ=536.70 K, and the weight loss was not significant at the melting temperature, indicating that Ac-Lys did not decompose when melted at a high temperature.

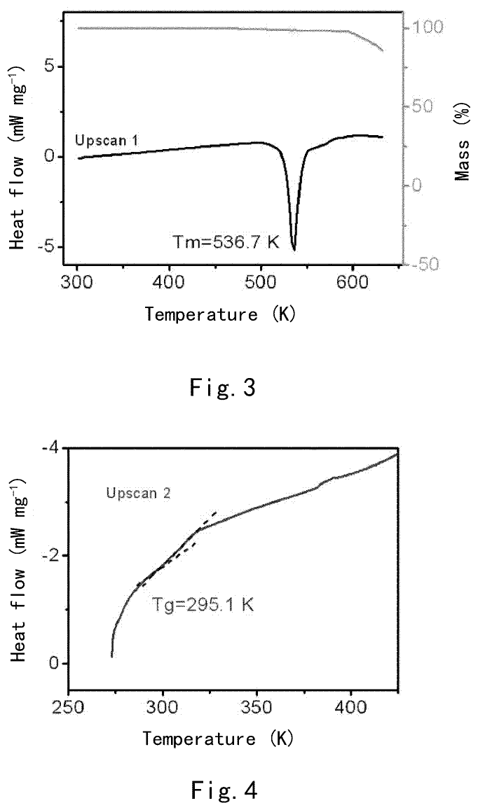

FIG. 3 shows the DSC diagram of the Ac-Lys glass prepared in Example 1, of which the glass transition temperature T_{g}=295.10 K.

### Example 2

A method for preparing phenylalanine-based peptide glass comprises the steps of:
(1) Weighing 50 mg of benzyloxycarbonyl-phenylalanyl-phenylalanyl (Z-Phe-Phe) powders, grinding in a mortar uniformly, then transferring to a crucible;
(2) Placing the crucible containing Z-Phe-Phe powders from step (1) in a heating device under N₂ atmosphere;
(3) Heating the crucible from room temperature to 500 K at a heating rate of 40 K min⁻¹ by conducting a heating treatment on the device from step (2), and maintaining at this temperature for 20 min;
(4) Lowering the temperature of the crucible to a temperature of 273.15 K at a cooling rate of 50 K min⁻¹ by conducting a cooling treatment on the device from step (3); and
(5) Transferring the sample from step (4) to an annealing furnace at a temperature of 283.15 K, and maintaining at this temperature for 10 min for conducting an annealing treatment on the glass, thereby obtaining Z-Phe-Phe glass.

FIG. 4 shows the H-NMR spectrum of the Z-Phe-Phe glass prepared in Example 2. Compared to the Z-Phe-Phe raw material, the peaks did not change significantly, indicating that the chemical composition of the raw material peptide molecules did not change when subjected to heating, melting and annealing treatments.

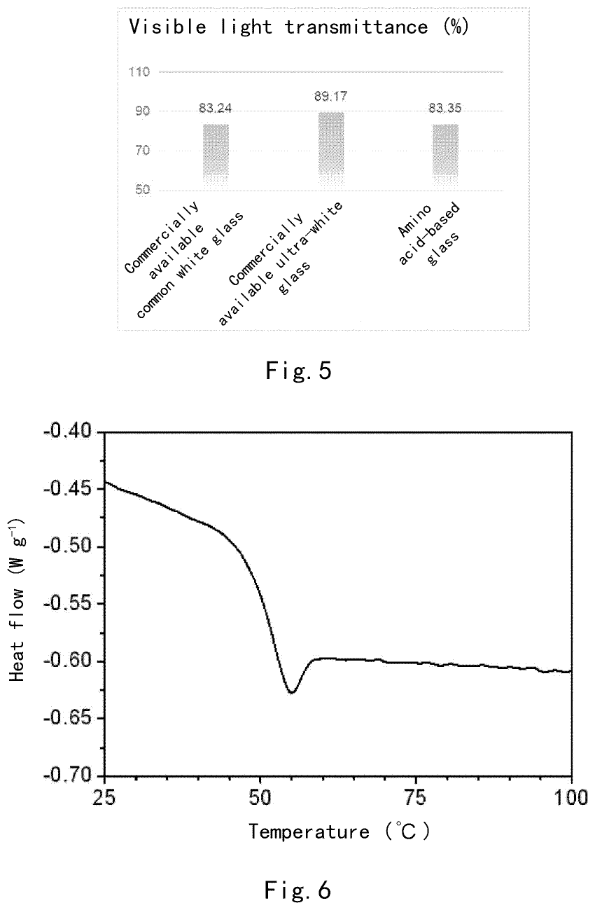

FIG. 5 shows the light transmittance of the Z-Phe-Phe glass prepared in Example 2, which is comparable to commercially available glass.

FIG. 6 shows the DSC diagram of the Z-Phe-Phe glass prepared in Example 2, of which the glass transition temperature T_{g}=320.75 K.

### Example 3

A method for preparing glycine-based glass comprises the steps of:
(1) Weighing 30 mg of N-tert-butoxycarbonyl-L-glycine (Boc-Gly) powders, grinding in a mortar uniformly, then transferring to a crucible;
(2) Placing the crucible containing Boc-Gly powders from step (1) in a heating device under N₂ atmosphere;
(3) Heating the crucible from room temperature to 600 K at a heating rate of 10 K min⁻¹ by conducting a heating treatment on the device from step (2), and maintaining at this temperature for 30 min;
(4) Lowering the temperature of the crucible to a temperature of 273.15 K at a cooling rate of 10 K min′ by conducting a cooling treatment on the device from step (3); and
(5) Transferring the sample from step (4) to an annealing furnace at a temperature of 283.15 K, and maintaining at this temperature for 30 min for conducting an annealing treatment on the glass, thereby obtaining Boc-Gly glass.

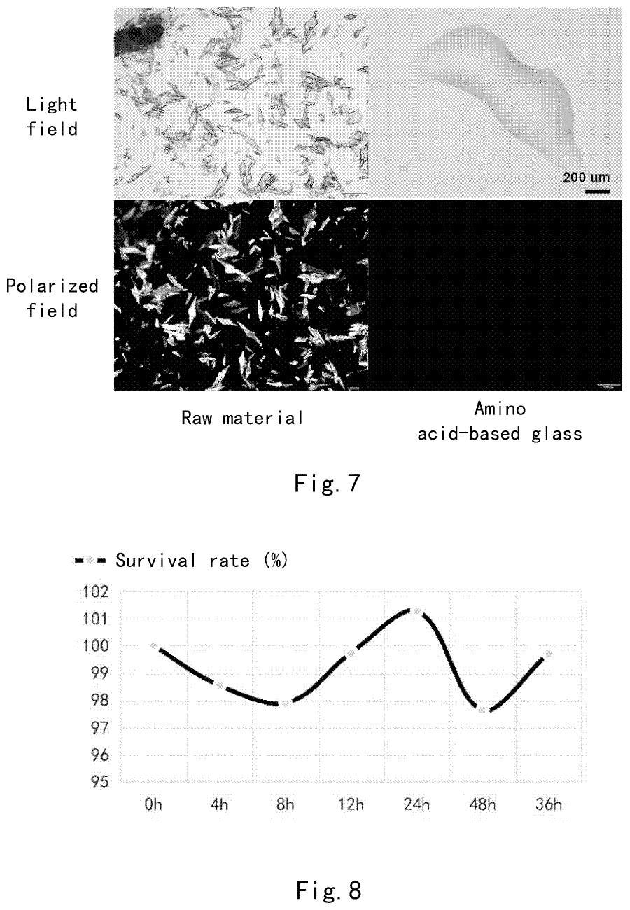

FIG. 7 shows the picture of Boc-Gly powders and Boc-Gly glass prepared in Example 3 under a polarizing microscope, demonstrating that the glass formed was amorphous.

FIG. 8 shows the test results for the biocompatibility of the Boc-Gly glass prepared in Example 3, in which the glass was processed into a square coating with a width of 2 cm, co-incubated with 3T3 cells, and the cell activity was tested by the MTT method. It is noted that the glass prepared in Example 3 did not dissolve in a neutral aqueous solution.

### Example 4

A method for preparing alanine-based glass comprises the steps of:
(1) Weighing 20 mg of N-tert-butoxycarbonyl-L-alanine (Boc-Ala) powders, grinding in a mortar uniformly, then transferring to a crucible;
(2) Placing the crucible containing Boc-Ala powders from step (1) in a heating device under N₂ atmosphere;
(3) Heating the crucible from room temperature to 650 K at a heating rate of 5 K by conducting a heating treatment on the device from step (2), and maintaining at this temperature for 5 min;
(4) Lowering the temperature of the crucible to a temperature of 273.15 K at a cooling rate of 20 K min′ by conducting a cooling treatment on the device from step (3); and
(5) Transferring the sample from step (4) to an annealing furnace at a temperature of 283.15 K, and maintaining at this temperature for 10 min for conducting an annealing treatment on the glass, thereby obtaining Boc-Ala glass.

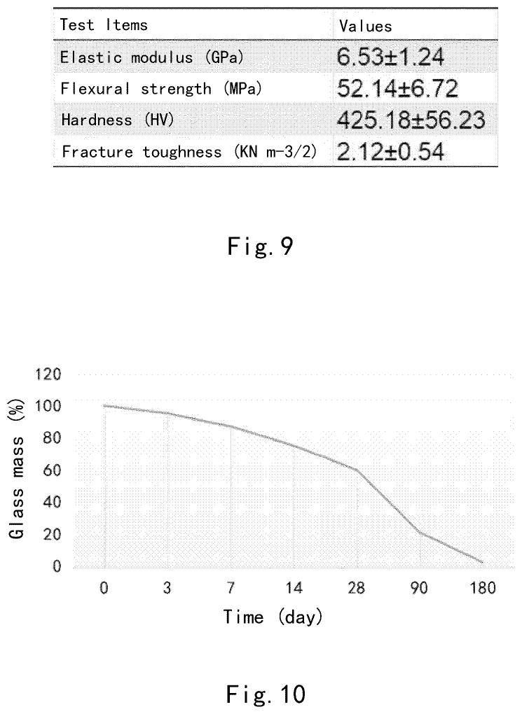

FIG. 9 shows the test results for mechanical properties of the Boc-Ala glass prepared in Example 4.

FIG. 10 shows the biodegradation curve of the Boc-Ala glass prepared in Example 4 in a compost soil sample, in which the initial mass of the glass sample was 42.58 mg.

### Example 5

A method for preparing phenylalanine and glutamic acid-based glass comprises the steps of:
(1) Weighing 10 mg of L-phenylalanine ethyl ester (Phe-OEt) powders and 10 mg of N-tert-butoxycarbonyl-L-glutamic acid dimethyl ester (Boc-Glu-dME) powders, grinding the mixture in a mortar uniformly, thereto adding 0.1 wt % of copper sulfate powders, and then grinding uniformly before transferring to a crucible;
(2) Placing the crucible containing the mixed amino acids from step (1) in a heating device under N₂ atmosphere;
(3) Heating the crucible from room temperature to 550 K at a heating rate of 10 K min′ by conducting a heating treatment on the device from step (2), and maintaining at this temperature for 10 min;
(4) Lowering the temperature of the crucible to a temperature of 273.15 K at a cooling rate of 10 K min′ by conducting a cooling treatment on the device from step (3); and
(5) Transferring the sample from step (4) to an annealing furnace at a temperature of 283.15 K, and maintaining at this temperature for 10 min for conducting an annealing treatment on the glass, thereby obtaining Phe-OEt/Boc-Glu-dME mixed glass.

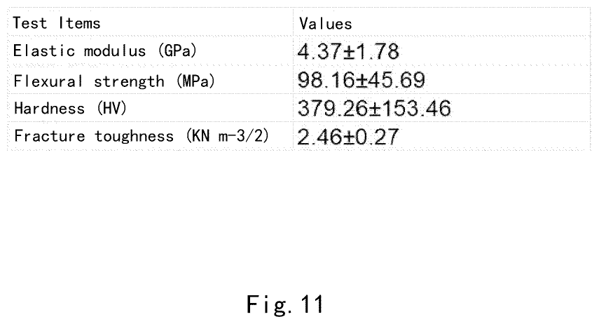

FIG. 11 shows the performance test results for the mixed glass prepared in Example 5.

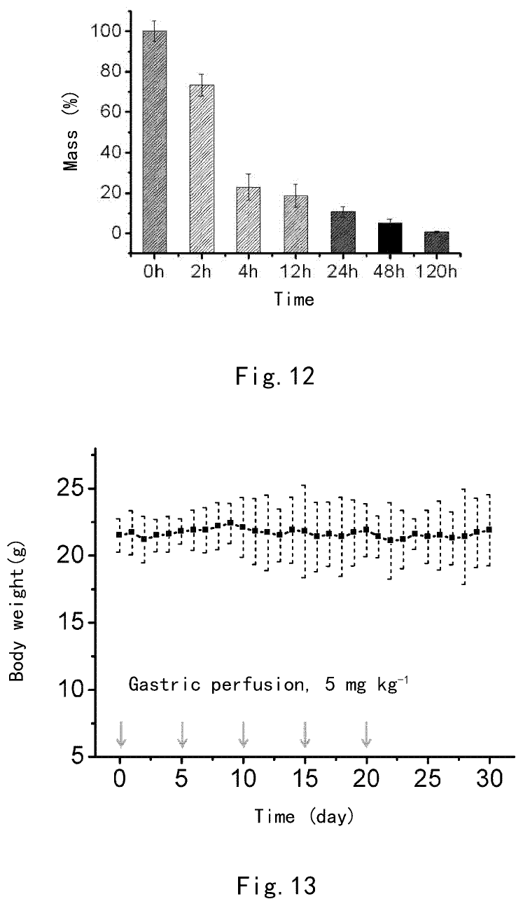

FIG. 12 shows the degradation of the mixed glass prepared in Example 5 in an artificial gastric juice (following the preparation method of the Chinese Pharmacopoeia).

FIG. 13 shows the body weight change of mice after gastric perfusion of the mixed glass prepared in Example 5. The cycle of gastric perfusion to mice is once every 5 days with a dose of 5 mg kg′ for an observation period of 30 days, resulting in the number of gastric perfusions of 5.

### Example 6

A method for preparing active peptide and amino acid derivative-based glass comprises the following steps:
(1) Weighing 10 mg of immunoactive peptide Val-Gln-Pro-Ile-Pro-Tyr powders and 10 mg of N-tert-butoxycarbonyl-L-arginine methyl ester (Boc-L-Arg-OMe) powders, grinding the mixture in a mortar uniformly, then transferring to a crucible;
(2) Placing the crucible containing the mixed powders from step (1) in a heating device under N₂ atmosphere;
(3) Heating the crucible from room temperature to 450 K at a heating rate of 10 K min⁻¹ by conducting a heating treatment on the device from step (2), and maintaining at this temperature for 20 min;
(4) Lowering the temperature of the crucible to a temperature of 273.15 K at a cooling rate of 10 K min⁻¹ by conducting a cooling treatment on the device from step (3); and
(5) Transferring the sample from step (4) to an annealing furnace at a temperature of 283.15 K, and maintaining at this temperature for 10 min for conducting an annealing treatment on the glass, thereby obtaining mixed glass.

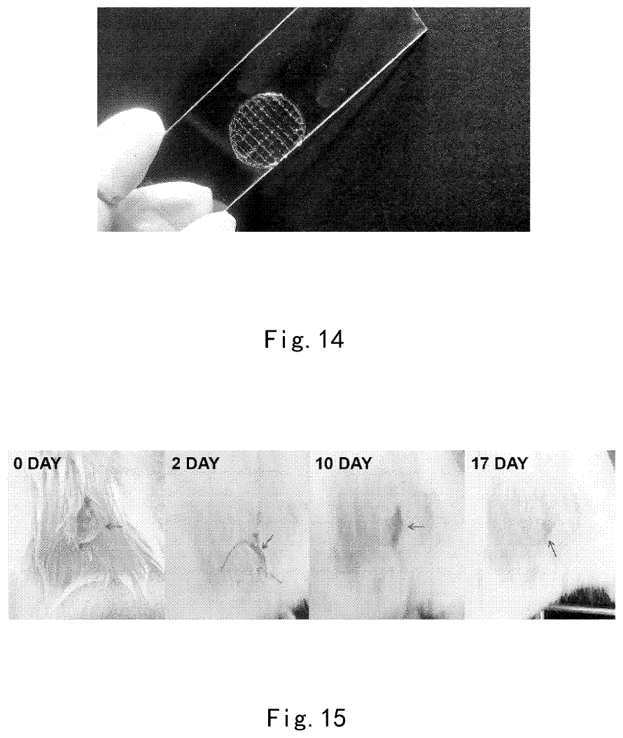

FIG. 14 shows the pattern printed with a 3D printing equipment from the mixed glass prepared in Example 6, in which the mixed powders were placed in the barrel of the 3D printing equipment, and the heating temperature was set to 450 K.

FIG. 15 shows the degradation of the mixed glass prepared in Example 6 after being implanted in an animal mouse model.

### Example 7

A method for preparing insulin-loaded amino acid-based glass comprises the following steps:
(1) Weighing 50 mg of immunoactive peptide Val-Gln-Pro-Ile-Pro-Tyr powders, grinding in a mortar uniformly, then transferring to a crucible;
(2) Placing the crucible containing the powders from step (1) in a heating device under N₂ atmosphere;
(3) Heating the crucible from room temperature to 450 K at a heating rate of 10 K min⁻¹ by conducting a heating treatment on the device from step (2), and maintaining at this temperature for 10 min, then cooling to 330 K;
(4) Weighing 5 mg of insulin powders, grinding in a mortar uniformly, then transferring to the crucible from step (3), stirring uniformly, and maintaining at this temperature for 10 min for melting;
(5) Lowering the temperature of the crucible to a temperature of 273.15 K at a cooling rate of 20 K min⁻¹ by conducting a cooling treatment on the device from step (4); and
(6) Transferring the sample from step (5) to an annealing furnace at a temperature of 283.15 K, and maintaining at this temperature for 20 min for conducting an annealing treatment on the glass, thereby obtaining the insulin-loaded amino acid-based glass.

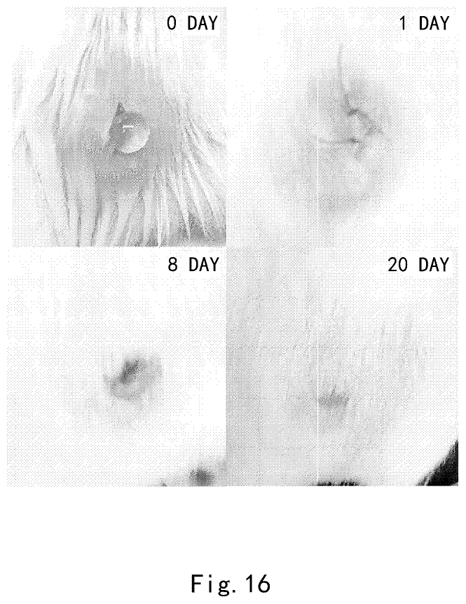

FIG. 16 shows the biodegradation of the insulin-loaded amino acid-based glass prepared in Example 7 after being subcutaneously implanted in mice over time.

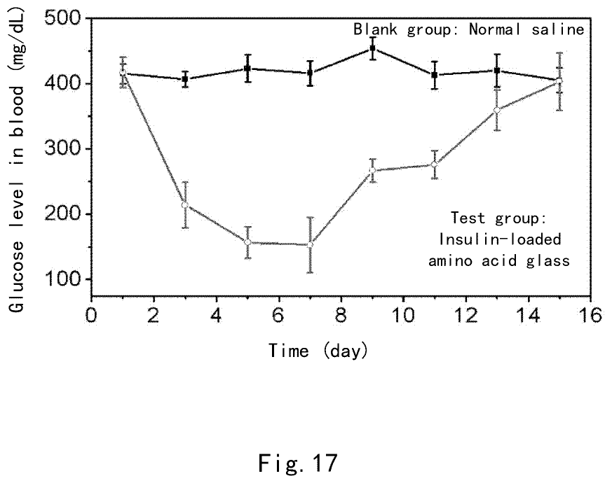

FIG. 17 shows the blood glucose change of diabetic mice after oral gastric perfusion of the insulin-loaded amino acid-based glass prepared in Example 7.

1. Amino acid-based glass, wherein the main raw material of the glass is one or more of an amino acid represented by formula (1), a peptide, their derivatives, or salts thereof, and the content of the main raw material in the glass is 70 wt % or more, preferably 80 wt % or more, further preferably 90 wt % or more; 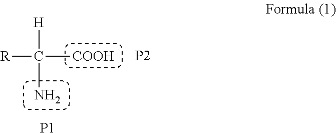
the amino acid is one or more selected from glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, selenocysteine, and pyrrolysine:
the peptide is a molecule formed by condensation of n above-mentioned amino acids via peptide bonds, where n≥2, preferably 2≤n≤10;
the derivatives of the amino acid or peptide are amino acids or peptides having protecting group(s) on the amino group P1 and/or the carboxyl group P2, wherein:
the protecting group on the amino group P1 is any one or more selected from Trt, Boc, Fmoc, Cbz/Z, Allyl, C₂-C₁₈acyl, benzoyl, and naphthoyl:
the protecting group on the carboxyl group P2 is any one or more selected from OFm, Othu, OBzl, OAll, OMe, and OEt:
the amino group P1 and the carboxyl group P2 are protected individually or simultaneously.

2. The amino acid-based glass according to claim 1, wherein the glass is prepared completely from the amino acid, peptide and their derivatives.

3. The amino acid-based glass according to claim 1, wherein the glass is prepared from a single molecule of the following: a single amino acid molecule, a single peptide molecule, a single amino acid derivative, or a single peptide derivative; or
the glass is composed of a combination of two or more molecules including:
a combination of amino acid molecules, a combination of peptide molecules, a combination of amino acid derivative molecules, a combination of peptide derivative molecules, a combination of amino acid molecules and peptide molecules, a combination of amino acid molecules and amino acid derivative molecules, a combination of amino acid molecules and peptide derivative molecules, a combination of peptide molecules and amino acid derivative molecules, a combination of peptide molecules and peptide derivative molecules, a combination of amino acid derivative molecules and peptide derivative molecules, a combination of amino acid molecules, peptide molecules and amino acid derivative molecules, a combination of amino acid molecules, peptide molecules and peptide derivative molecules, a combination of amino acid molecules, amino acid derivative molecules and peptide derivative molecules, and a combination of amino acid molecules, peptide molecules, amino acid derivative molecules and peptide derivative molecules.

4. The amino acid-based glass according to claim 1, wherein the glass further comprises auxiliary raw materials selected from one of or a mixture of two or more of clarifiers, fluxes, opacifiers, and colorants.

5. The amino acid-based glass according to claim 1, wherein the glass has a hardness of between 420˜550 HV, preferably between 500˜550 HV; and the glass has a transparency of 30% or more, preferably 60% or more, further preferably 80%-91%.

6. The amino acid-based glass according to claim 1, wherein the glass has a brittleness index (m) of between 10˜100, preferably between 20˜50.

7. A preparation method for the amino acid-based glass according to claim 1, comprising the steps of:
raising the temperature of raw materials to a temperature higher than the melting point (Tₘ) under an inert gas atmosphere, and maintaining at this temperature for a period of time,
lowering the temperature to below room temperature, and
transferring the temperature-lowered sample to an annealing furnace for annealing treatment.

8. The preparation method according to claim 7, wherein the temperature higher than the melting point is a temperature higher than the melting point by 5˜200 K, preferably higher than the melting point by 10˜50 K: and the time for maintaining is 5 min˜1 h, preferably 15˜30 min.

9. The preparation method according to claim 8, wherein the temperature for the annealing treatment is a temperature lower than the glass transition temperature (T_{g}) by 20˜100 K, preferably 20˜50 K; and the time for the annealing treatment is 5 min˜3 h, preferably 15 min˜1 h.

10. Use of the amino acid-based glass according to claim 1, including the use for additive manufacturing, composting, tissue engineering, tooth or bone repair, drug sustained-release, cell or protein sequestration, optical fiber communication, coatings, and precision instruments.
